(19) United States
(12) Patent Application Publication
(10) Pub. No.: US 2004/0029269 A1
(43) Pub. Date: Feb. 12, 2004

(54) Promoter-based isolation, purification, expansion, and transplantation of neuronal progenitor cells, oligodendrocyte progenitor cells, or neural stem cells from a population of embryonic stem cells

(76) Inventors: Steven A. Goldman, South Salem, NY (US); Neeta Singh Roy, New York, NY (US)

Correspondence Address: Michael L. Goldman, NIXON PEABODY LLP, Clinton Square, P.O. Box 31051, Rochester, NY, 14603-1051, US

(21) Appl. No.: 10/430,822

(22) Filed: May 6, 2003

Related U.S. Application Data
(60) Provisional application No. 60/378,802, filed on May 7, 2002.

(51) Int. Cl. C12N 5/08

(52) U.S. Cl. 435/368

(57) ABSTRACT

The present invention relates to a method of isolating neuronal progenitor cells, oligodendrocyte progenitor cells, or neural stem cells from a population of embryonic stem cells. This method comprises selecting a promoter which functions only in neuronal progenitor cells, oligodendrocyte progenitor cells, or neural stem cells and introducing a nucleic acid molecule encoding a marker protein under control of said promoter into the population of embryonic stem cells. The population of embryonic stem cells are then differentiated to produce a mixed population of cells comprising neuronal progenitor cells, oligodendrocyte progenitor cells, or neural stem cells. The neuronal progenitor cells, oligodendrocyte progenitor cells, or neural stem cells are then allowed to express the marker protein. Cells expressing the marker protein are separated from the mixed population of cells, where the separated cells are neuronal progenitor cells, oligodendrocyte progenitor cells, or neural stem cells. In an alternative embodiment, the embryonic stem cells are differentiated before the nucleic acid is introduced. The present invention also relates to the resulting neuronal progenitor cells, oligodendrocyte progenitor cells, or neural stem cells themselves.

This application claims the benefit of U.S. Provisional Patent Application Serial No. 60/378,802, filed May 7, 2002.

### FIELD OF THE INVENTION

The present invention is directed to promoter-based isolation, purification, expansion, and transplantation of neuronal progenitor cells, oligodendrocyte progenitor cells, or neural stem cells from a population of embryonic stem cells.

### BACKGROUND OF THE INVENTION

The damaged adult mammalian brain is incapable of significant structural self-repair. Terminally differentiated neurons are incapable of mitosis, and compensatory neuronal production has not been observed in any mammalian models of structural brain damage (Korr, “Proliferation of Different Cell Types in the Brain,” Adv. Anat. Embryol. Cell. Biol., 61:1-72 (1980) and Sturrock, “Changes in Cell Number in the Central Canal Ependyma and in the Dorsal Grey Matter of the Rabbit Thoracic Spinal Cord During Fetal Development,” J. Anat., 135:635-647 (1982)). Although varying degrees of recovery from injury are possible, this is largely because of synaptic and functional plasticity rather than the frank regeneration of neural tissues. The lack of structural plasticity of the adult brain is partly because of its inability to generate new neurons, a limitation that has severely hindered the development of therapies for neurological injury or degeneration. Indeed, the inability to replace or regenerate damaged or dead cells continues to plague neuroscientists, neurologists, and neurosurgeons who are interested in treating the injured brain. During the last several years, however, a considerable body of evidence has evolved that suggests a marked degree of cellular plasticity in the adult as well as in the developing CNS. In particular, recent work on neural progenitor cells, derived from both embryos and adults, has suggested strategies for directed neuronal regeneration and structural brain repair. These include the use of neural stem cells which are the multipotential progenitors of neurons and glia that are capable of self-renewal (Davis et al., “A Self-Renewing Multipotential Stem Cell in Embryonic Rat Cerebral Cortex,” Nature, 372:263-266 (1994); Gritti et al., “Multipotential Stem Cells from the Adult Mouse Brain Proliferate and Self-Renew in Response to Basic Fibroblast Growth Factor,” J. Neurosci., 16:1091-1100 (1996); Kilpatrick et al., “Cloning and Growth of Multipotential Neural Precursors: Requirements for Proliferation and Differentiation,” Neuron, 10:255-265 (1993); Morshead et al., “Neural Stem Cells in the Adult Mammalian Forebrain: A Relatively Quiescent Subpopulation of Subependymal Cells,” Neuron, 13:1071-1082 (1994); Stemple et al., “Isolation of a Stem Cell for Neurons and Glia from the Mammalian Neural Crest,” Cell 71:973-985 (1992); Kirschenbaum, et al., “In vitro Neuronal Production by Precursor Cells Derived from Adult Human Brain,” Cereb. Cortex 4:576:89 (1994); Pincus, et al., “FGF2/BDNF-Associated Maturation of New Neurons Generated from Adult Human Subependymal Cells,” Ann. Neurol. 43:576-85 (1998); Roy et al., “In vitro Neurogenesis by Neural Progenitor Cells Isolated from the Adult Human Hippocampus,” Nature Med. 6:271-77 (2000); Roy et al., “Promoter-Targeted Selection and Isolation of Neural Progenitor Cells from the Adult Human Ventricular Zone,” J. Neurosci. Res. 59:321-31 (2000); Keyoung et al., “Specific Identification, Selection, and High-Yield Extraction of Neural Stem Cells from Fetal Human Brain,” Nature Biotechnol. 19:843-50 (2001); Nunes, et. al., “Identification and Isolation of Multipotent Neural Progenitor Cells from the Subcortical White Matter of the Adult Human Brain,” Nature Med. 9:239-47 (2003); and Weiss et al., “Is There a Neural Stem Cell in the Mammalian Forebrain?,” Trends Neurosci., 19:387-393 (1996)).

In the adult human brain, both neuronal and oligodendroglial precursors have been identified as well, and methods for their harvest and enrichment have been established. Neural precursors have several characteristics that make them ideal vectors for brain repair. They may be expanded in tissue culture, providing a renewable supply of material for transplantation. Moreover, progenitors are ideal for genetic manipulation and may be engineered to express exogenous genes for neurotransmitters, neurotrophic factors, and metabolic enzymes (reviewed in Goldman, “Adult Neurogenesis: From Canaries to the Clinic,” J. Neurobiol. 36:267-86 (1998); Pincus et al., “Fibroblast Growth Factor-2/Brain-Derived Neurotrophic Factor-Associated Maturation Of New Neurons Generated From Adult Human Subependymal Cells,” Ann. Neurol., 43(5):576-85 (1998); and Goldman et al., “Strategies Utilized by Migrating Neurons of the Postnatal Vertebrate Forebrain,” Trends in Neurosci. 21(3):107-14 (1998)).

In embryonic neurogenesis, the proliferation of neuronal precursors takes place at the surface of the central canal lining the neural tube (Jacobson, “Developmental Neurobiology” New York: Plenum Press (1991)). The central canal ultimately forms the ventricular system of the adult. This neurogenic layer is referred to as the ventricular/subventricular zone in development, and the ependymal/subependymal zone (SZ) in adults (Boulder Committee, “Embryonic Vertebrate Central Nervous System: Revised Terminology,” Anat. Rec., 166:257-261 (1970)). In development, mitogenesis in the ventricular/subventricular zone is followed by the migration of newly generated neurons and glia along radial guide fibers into the brain parenchyma, including that of the cortical plate (LaVail et al., “The Development of the Chick Optic Tectum: II-Autoradiographic Studies,” Brain Res., 28:421-441 (1971); Rakic, “Guidance of Neurons Migrating to the Fetal Monkey Neocortex,” Brain Res., 33:471-476 (1971); Rakic, “Neurons in Rhesus Monkey Visual Cortex: Systematic Relation Between the Time of Origin and Eventual Disposition,” Science, 183:425-427 (1974); and Sidman et al., “Neuronal Migration, with Special Reference to Developing Human Brain: A Review,” Brain Res., 62:1-35 (1973)).

Human embryonic stem cells (hES cells) can generate many if not all major cellular phenotypes. These include neurons and glia and, by inference, their parental neural stem or progenitor cells. However, achieving purified preparations of neural stem cells from mixed populations of hES cells has hitherto proven an intractable task. Although populations of hES cells can be directed toward one phenotype or another, the resultant cultures are still highly mixed, almost invariably with at least 10% of cells developing along lineages other than the desired phenotype. These contaminants are unacceptable, as undesired phenotypic differentiation can lead to inappropriate, and perhaps dangerous, ectopic cell development, and also since residual undifferentiated hES cells can be tumorigenic upon implantation.

The present invention is directed to overcoming these and other deficiencies in the art.

### SUMMARY OF THE INVENTION

The present invention relates to a method of isolating neuronal progenitor cells, oligodendrocyte progenitor cells, or neural stem cells from a population of embryonic stem cells. This method comprises selecting a promoter which functions only in neuronal progenitor cells, oligodendrocyte progenitor cells, or neural stem cells and introducing a nucleic acid molecule encoding a marker protein under control of said promoter into the population of embryonic stem cells. The population of embryonic stem cells are then differentiated to produce a mixed population of cells comprising neuronal progenitor cells, oligodendrocyte progenitor cells, or neural stem cells. The neuronal progenitor cells, oligodendrocyte progenitor cells, or neural stem cells are then allowed to express the marker protein. Cells expressing the marker protein are separated from the mixed population of cells, where the separated cells are neuronal progenitor cells, oligodendrocyte progenitor cells, or neural stem cells.

In another embodiment, the present invention relates to a method of isolating neuronal progenitor cells, oligodendrocyte progenitor cells, or neural stem cells from a population of embryonic stem cells by providing a population of embryonic stem cells and differentiating the population of embryonic stem cells to produce a mixed population of cells comprising neuronal progenitor cells, oligodendrocyte progenitor cells, or neural stem cells. A promoter which functions only in said neuronal progenitor cells, oligodendrocyte progenitor cells, or neural stem cells is selected and a nucleic acid molecule encoding a marker protein under control of the promoter is introduced into the mixed population of cells. The neuronal progenitor cells, oligodendrocyte progenitor cells, or neural stem cells are then allowed to express the marker protein. Cells expressing the marker protein are separated from the mixed population of cells, where the separated cells are neuronal progenitor cells, oligodendrocyte progenitor cells, or neural stem cells.

Another aspect of the present invention is an enriched or purified preparation of isolated oligodendrocyte progenitor cells derived from embryonal stem cells.

A further embodiment of the present invention relates to an enriched or purified preparation of isolated neuronal progenitor cells derived from embryonal stem cells.

Yet an additional aspect of the present invention is directed to an enriched or purified preparation of isolated neural stem cells derived from embryonal stem cells.

To selectively identify and extract neural stem cells from larger populations of human embryonic stem cells, hES cells were infected with both plasmids and adenoviruses bearing the gene for green fluorescence protein (“GFP”), placed under the control of the second intronic enhancer of the nestin gene (“E/nestin”). This is a regulatory region that is selectively activated in uncommitted neuroepithelial cells. The cells were then sorted via fluorescence-activated cell sorting (“FACS”) on the basis of E/nestin-driven GFP expression. The isolated and purified E/nestin:enhanced GFP (“EGFP”)-sorted cells were multipotent: Limiting dilution, with clonal expansion as neurospheres, revealed that each phenotype was able to both self-renew and co-generate neurons and glia. These hES-derived human neural stem cells could be maintained in continuously expanding culture, in which they generated both neurons and glia at all timepoints during in vitro expansion. Furthermore, the cells engrafted well upon transplantation, with effective integration as neurons and glial cells. Thus, promoter-specified FACS was successfully used to prepare highly enriched populations of implantable neural stem cells from cultures of human embryonic stem cells.

The isolation of neural stem cells from embryonic stem cells has been a major issue in hES cell-based therapy, and has already been approached through several means intended to enrich these cells (Reubinoff et al., “Neural Progenitors from Human Embryonic Stem Cells,” Nature Biotechnol. 19:1134-1140 (2001) and Zhang et al., “In Vitro Differentiation of Transplantable Neural Precursors from Human Embryonic Stem Cells,” Nature Biotechnol. 19:1129-1133 (2001), which are hereby incorporated by reference in their entirety). However, neither of the currently described strategies permits as high a degree of purity as the present invention, because neither excludes non-neural stem cells from the enriched population. The danger of this is that any remnant undifferentiated hES cells in an incompletely enriched pool of neural stem cells may form undesired cell types after implantation, or may become teratomas, tumors of embryonic stem cells. The present invention provides for, the positive selection and differential extraction of neural stem cells from either an untreated and mixed population of ES cells and their derivatives, or from an already-enriched population of neural stem cells. The purity of the resultant cell populations is much higher than previously available techniques, readily permitting >99% purification, and >99.9% provided a decrement in yield and sort speed is accepted.

Highly enriched or purified populations of neural stem cells may be used for transplantation into a variety of conditions of brain and spinal cord disease and injury (Gage, “Mammalian Neural Stem Cells,” Science 287:1433-1438 (2000), which is hereby incorporated by reference in its entirety). These cells may differentiate into neurons, oligodendrocytes, and astrocytes, and as such may reconstitute the lost cellular elements of the injured brain and spinal cord (Goldman, “Adult Neurogenesis: From Canaries to the Clinic,” J. Neurobiology 36:267-286 (1998); Pincus et al., “Neural Stem and Progenitor Cells: A Strategy for Gene Therapy and Brain Repair,” Neurosurgery 42:858-868 (1998); Svendsen et al., “Neural Stem Cells in the Developing Central Nervous System: Implications for Cell Therapy Through Transplantation,” Prog. Brain Res. 127:13-34 (2000); and Svendsen et al., “New Prospects for Human Stem-Cell Therapy in the Nervous System,” Trends Neurosci. 22:357-364 (1999), which are hereby incorporated by reference in their entirety). Their implantation into diseased areas may mediate structural regeneration and repair. For instance, neural stem cell implants, or implants of their derived neuronal and glial progenitors, have been found to reconstitute brain tissues lost to stroke and injury (Snyder et al., “Multipotent Neural Precursors Can Differentiate Toward Replacement of Neurons Undergoing Targeted Apoptotic Degeneration in Adult Mouse Neocortex,” Proc. Natl. Acad. Sci. USA 94:11663-11668 (1997), which is hereby incorporated by reference in its entirety), and to remyelinate regions of the brain demyelinated in either chemical (Windrem et al., “Progenitor Cells Derived from the Adult Human Subcortical White Matter Disperse and Differentiate as Oligodendrocytes Within Demyelinated Regions of the Rat Brain,” J. Neurosci. Res. 69:966-975 (2002), which is hereby incorporated by reference in its entirety) or autoimmune demyelination (Pluchino et al., “Injection of Adult Neurospheres Induces Recovery in a Chronic Model of Multiple Sclerosis,” Nature 422:688-694 (2003), which is hereby incorporated by reference in its entirety). In addition, neural stem cells are highly migratory in perinatal brain and, as a result, may also be used to replete deficient or mutated enzymes in hereditary and metabolic diseases (Yandava et al., “Global Cell Replacement is Feasible Via Neural Stem Cell Transplantation: Evidence from the Dysmyelinated Shiverer Mouse Brain,” Proc. Natl. Acad. Sci. USA 96:7029-7034 (1999), which is hereby incorporated by reference in its entirety). To date, most experimental therapeutic studies of neural stem and progenitor cell engraftment have been done using fetal or adult tissues as the sources of neural stem and progenitor cells. These approaches have required the constant acquisition of new fetal and adult tissues, from both animal and human donors. The latter is particularly problematic as a source, since human tissue-derived cells, whether of fetal or adult origin (Keyoung et al., “Specific Identification, Selection and Extraction of Neural Stem Cells from the Fetal Human Brain,” Nature Biotech. 19:843-850 (2001); Pincus et al., “Fibroblast Growth Factor-2/Brain-Derived Neurotrophic Factor-Associated Maturation of New Neurons Generated from Adult Human Subependymal Cells,” Ann. Neurol. 43:576-585 (1998); Roy et al., “Promoter-Targeted Selection and Isolation of Neural Progenitor Cells from the Adult Human Ventricular Zone,” J. Neurosci. Res. 59:321-331 (2000); and Roy et al., “In Vitro Neurogenesis by Progenitor Cells Isolated from the Adult Human Hippocampus,” Nature Med. 6:271-277 (2000), which are hereby incorporated by reference in their entirety), are difficult to obtain and more difficult to standardize and scale up. In contrast, hES cells permit the ready acquisition, scalable expansion and directed differentiation of neural stem and progenitor cells. Neural stem and progenitor cells derived from ES cells, of both murine and human origin, have already been found to be capable of both remyelination, in the case of ES-derived glial and oligodendrocytes, and dopaminergic replenishment in experimental Parkinson's disease, in the case of ES-derived midbrain dopaminergic neurons. By now achieving the purification of neural stem cells from ES cells culture, the present invention increases substantially both the reliability and safety of their use and may, therefore, obviate the need for human tissue acquisition. Thus, the high-grade enrichment to purity of neural stem from human ES cells has been an important challenge in the field, which is addressed by the current invention.

### BRIEF DESCRIPTION OF THE DRAWINGS

FIGS. 1A-B show nestin-expressing cells arise at the differentiating margins of human embryonic stem cells. These cells are maintained in Knockout DMEM (Gibco) supplemented with 20% serum and express GFP within 3 days of infection by adenoviral (“Ad”)E/Nestin EGFP.

FIGS. 2A-B show E/nestin:EGFP recognizes only a minority of human embryonic stem cells 9 days after passage and 7 days post-AdE/nestin:EGFP infection. The human embryonic stem cells are induced to form embryonic bodies in Knockout DMEM (Gibco) supplemented with 20% PBS and continue to express GFP 7 days after infection by AdE/Nestin EGFP.

FIGS. 3A-B show that FACS selects a distinct population of E/nestin-driven GFP⁺. Flow cytometric analysis of ADE/Nestin:EGFP infected human embryonic stem cells showed that the EGFP expressing population constituted 5.67±1.8% (mean±SD, n=4 samples) of the total cell population.

FIGS. 4A-B show FACS results which suggest several size ranges of E/nestin-driven GFP⁺ cells. Profiles of forward scatter (“FSC”) v. fluorescence intensity (“FL1”) reveal the presence of two populations of nestin⁺ progenitor cells.

FIGS. 5A-B show AdE/Nestin:EGFP-induced human embryonic stem cells can be extracted to near homogeneity by FACS. Following 5 days after, FACS, in knock out-DMEM supplemented with 20% FBS and RA, the sorted cells start to form spheres and continue to express nestin-driven GFP.

FIGS. 6A-B show E/nestin:EGFP-sorted -human embryonic stem cells differentiate largely as neurons and glia with FIG. 6A showing the results 6 days after treatment with brain derived neurotrophic factor (“BDNF”)/neurotrophin-3 (“NT-3”) and FIG. 6B showing βIII-tubulin treatment. Following differentiation in Neurobasal medium supplemented with B27 (Gibco), NT3, and BDNF and on polyornithine/fibronectin coated plates for 5 days, βIII-tubulin expression was observed by most of the nestin-sorted cells, indicating their neuronal differentiation and maturation.

FIG. 7 shows highly enriched populations of neurons can be derived from human embryonic stem cells sorted by FACS on the basis of E/nestin-driven GFP where the βIII-tubulin promoter is used.

FIGS. 8A-B show adenoviral with Tα1 tubulin promoter (“AdTα”):human embryonic stem cells recognize neuronal progenitor cells within mixed cultures of human embryonic stem cells.

FIGS. 9A-B show AdTα:human embryonic stem cells recognize a population of neuronally-differentiating human embryonic stem cells. Human embryonic stem cells maintained in KO-DMEM supplemented with 20% KO-serum exhibited GFP expression by neuronal progenitor cells within 3 days of infection with AdP/Tα1:hGFP.

FIG. 10 is a schematic drawing showing the selection of neural stem cells from a population of embryonic stem cells.

The figure titles and legends for FIGS. 11-17 are on the figure prinouts it self. Here it is once again,

FIGS. 11A-D demonstrate that lentivirus (“Lenti”)-E/Nestin:EGFP expression can be seen at the differentiating margins and centers of hES colonies. hES cells maintained in Knockout DMEM/Knockout replacement serum (Gibco) were infected with Lenti-E/Nestin:EGFP virus. EGFP expression was observed 3-4 days after infection. Typically EGFP expression was observed at the edges (FIGS. 11A and B) or center (FIGS. 11C and D) of the hES colonies.

FIGS. 12A-D show that EGFP expression by Lenti-E/Nestin:EGFP infected hES cells continues through several generations. Lenti-E/Nestin:EGFP-positive cells maintained their EGFP expression through several generations (at passage 2 in FIGS. 12A-D). The EGFP expression profile was replicated in every passage, with EGFP expression being limited to the differentiating edges (FIGS. 12A and B) and centers (FIGS. 12C and D).

FIGS. 13A-D demonstrate that EGFP expression by Lenti-E/Nestin:EGFP infected hES cells continues through several generations without loss in intensity of EGFP expression. Lenti-E/Nestin:EGFP-positive cells continue to maintain their EGFP expression intensity and expression profiles (FIGS. 13A and C, seen at passage three).

FIGS. 14A-B show that Lenti-E/Nestin:EGFP expressing cells constitute a large proportion of the hES population. Flow cytometeric analysis showed that an average of 12.5% (FIGS. 14A and B) of the Lenti-E/Nestin:EGFP infected hES cells expressed EGFP.

FIGS. 15A-B show that FACS purified Lenti-E/Nestin:EGFP-expressing cells on induction of differentiation gave rise to neurons and glia. When sorted cells are cultured sequentially in the presence of DMEM/F12 supplemented with B-27, basic fibroblast growth factor (“bFGF”), epidermal growth factor (“EGF”), platelet derived growth factor (“PDGF”), and insulin-like growth factor (“IGF”) followed by BDNF/NT3, majority of the cells differentiated as 1III-tubulin expressing neurons (FIG. 15A) and some as GFAP (FIG. 15A) expressing glia.

FIGS. 16A-F demonstrate that Lenti-Ta1:hGFP recognizes neuronal progenitors in mixed hES cell cultures. hES cell cultures infected with Lenti-Tα1:hGFP virus start expressing GFP 3-4 days post-infection. GFP expression is limited to the nucleus (FIGS. 16A, C and E) and observed in cells either in the differentiating center of the hES colonies (FIGS. 16A and E) or in clusters of cells undergoing spontaneous differentiation (FIGS. 16C and D, arrow). From these differentiating clusters, neurons can be seen migrating out (FIG. 16C and D, arrow head).

FIGS. 17A-B show that Lenti-Tα1:hGFP is expressed by a significant proportion of the hES population. Flow cytometery analysis of Lenti-Tα1:hGFP infected cells indicate that around 7.32% of the total hES cells express GFP driven by the Tα1 promoter.

### DETAILED DESCRIPTION OF THE INVENTION

As used herein, the term “isolated” when used in conjunction with a nucleic acid molecule refers to: 1) a nucleic acid molecule which has been separated from an organism in a substantially purified form (i.e. substantially free of other substances originating from that organism), or 2) a nucleic acid molecule having the same nucleotide sequence but not necessarily separated from the organism (i.e. synthesized or recombinantly produced nucleic acid molecules).

“Enriched” refers to a cell population that is at least 90% pure with respect to the index phenotype, regardless of its initial incidence in the population from which it was derived. “Purified” refers to a cell population at least 99% pure with respect to the index phenotype, regardless of its initial incidence in the reference population.

The present invention relates to a method of isolation neuronal progenitor cells, oligodendrocyte progenitor cells, or neural stem cells from a population of embryonic stem cells. This method comprises selecting a promoter which functions only in neuronal progenitor cells, oligodendrocyte progenitor cells, or neural stem cells and introducing a nucleic acid molecule encoding a marker protein under control of said promoter into the population of embryonic stem cells. The population of embryonic stem cells are then differentiated to produce a mixed population of cells comprising neuronal progenitor cells, oligodendrocyte progenitor cells, or neural stem cells. The neuronal progenitor cells, oligodendrocyte progenitor cells, or neural stem cells are then allowed to express the marker protein. Cells expressing the marker protein are separated from the mixed population of cells, where the separated cells are neuronal progenitor cells, oligodendrocyte progenitor cells, or neural stem cells.

The process of selecting progenitors to select a particular cell type from a population of embryonic stem cells involves using a promoter that functions in the progenitor cells and a nucleic acid encoding a marker protein, as described in U.S. Pat. No. 6,245,564 to Goldman et. al., which is hereby incorporated by reference in its entirety. In particular, this involves providing a population of embryonic stem cells which population includes progenitor cells of a particular cell type and selecting a promoter which functions in the desired cells. A nucleic acid molecule encoding a marker protein under control of said promoter is introduced into the population of embryonic stem cells, and the population of progenitor cells is allowed to express the marker protein. The cells expressing the marker protein are separated from the population of cells, with the separated cells being the progenitor cells.

Any cell which one desires to separate from a plurality of cells can be selected in accordance with the present invention, as long as a promoter specific for the chosen cell is available. “Specific”, as used herein to describe a promoter, means that the promoter functions only in the chosen cell type. A chosen cell type can refer to different types of cells or different stages in the developmental cycle of a progenitor cell. For example, the chosen cell may be committed to a particular adult neural cell phenotype and the chosen promoter only functions in that neural progenitor cell; i.e. the promoter does not function in adult neural cells. Although committed and uncommitted neural progenitor cells may both be considered neural progenitor cells, these cells are at different stages of neural progenitor cell development and can be separated and immortalized according to the present invention if the chosen promoter is specific to the particular stage of the neural progenitor cell. Those of ordinary skill in the art can readily determine a cell of interest to select based on the availability of a promoter specific for that cell of interest.

Where neuronal progenitor cells are to be separated from the population of embryonic stem cells, the promoter can be a Tα1 tubulin promoter (Gloster et al., J. Neurosci. 14:7319-30 (1994), which is hereby incorporated by reference in its entirety), a Hu promoter (Park et al., “Analysis of Upstream Elements in the HuC Promoter Leads to the Establishment of Transgenic Zebrafish with Fluorescent Neurons,” Dev. Biol. 227(2): 279-93 (2000), which is hereby incorporated by reference in its entirety), an ELAV promoter (Yao et al., “Neural Specificity of ELAV Expression: Defining a Drosophila Promoter for Directing Expression to the Nervous System,” J. Neurochem. 63(1): 41-51 (1994), which is hereby incorporated by reference in its entirety), a MAP-1B promoter (Liu et al., Gene 171:307-08 (1996), which is hereby incorporated by reference in its entirety, or a GAP-43 promoter). See U.S. Pat. No. 6,245,564 to Goldman et. al., which is hereby incorporated by reference in its entirety.

For separation of oligodendrocyte progenitor cells from the population of embryonic stem cells, the promoter is a CNP promoter (Scherer et al., Neuron 12:1363-75 (1994), which is hereby incorporated by reference in its entirety), an NCAM promoter (Holst et al., J. Biol. Chem. 269:22245-52 (1994), which is hereby incorporated by reference in its entirety), a myelin basic protein promoter (Wrabetz et al., J. Neurosci. Res. 36:455-71 (1993), which is hereby incorporated by reference in its entirety), a JC virus minimal core promoter (Krebs et al., J. Virol. 69:2434-42 (1995), which is hereby incorporated by reference in its entirety), a myelin-associated glycoprotein promoter (Laszkiewicz et al., “Structural Characterization of Myelin-associated Glycoprotein Gene Core Promoter,” J. Neurosci. Res. 50(6): 928-36 (1997), which is hereby incorporated by reference in its entirety), or a proteolipid protein promoter (Cook et al., “Regulation of Rodent Myelin Proteolipid Protein Gene Expression,” Neurosci. Lett. 137(1): 56-60 (1992); Wight et al., “Regulation of Murine Myelin Proteolipid Protein Gene Expression,” J. Neurosci. Res. 50(6): 917-27 (1997); and Cambi et al., Neurochem. Res. 19:1055-60 (1994), which are hereby incorporated by reference in their entirety). See U.S. Pat. No. 6,245,564 to Goldman et. al., which is hereby incorporated by reference in its entirety.

Neural stem cells are separated from the population of embryonic stem cells with the musashi promoter or the nestin enhancer. See WO 01/46384 to Goldman et al.; Keyoung et al., “Specific Identification, Selection and High-Yield Extraction of Neural Stem Cells from the Fetal Human Brain,” Nature Biotech. 19:843-50 (2001); Sawamoto et al., “Direct Isolation of Committed Neuronal Progenitor Cells from Transgenic Mice Co-Expressing Spectrally-Distinct Fluorescent Proteins Regulated by Stage-Specific Neural Promoters,” J. Neurosci Res. 65:220-27 (2001); Kawaguchi et al., “Nestin-EGFP Transgenic Mice: Visualization of the Self-Renewal and Multiplicity of CNS Stem Cells,” Molec. Cell Neurosci. 17:259-73 (2001); Sawamoto et al., “Generation of Dopaminergic Neurons in the Adult Brain from Mesencephalic Precursor Cells Labeled with a Nestin-GFP Transgene,” J. Neurosci. 21:3895-903 (2001), which are hereby incorporated by reference in their entirety.

Having determined the cell of interest and selected a promoter specific for the cell of interest, a nucleic acid molecule encoding a protein marker, preferably a green fluorescent protein under the control of the promoter is introduced into a plurality of cells to be sorted.

The isolated nucleic acid molecule encoding a green fluorescent protein can be deoxyribonucleic acid (DNA) or ribonucleic acid (RNA, including messenger RNA or mRNA), genomic or recombinant, biologically isolated or synthetic. The DNA molecule can be a cDNA molecule, which is a DNA copy of a messenger RNA (mRNA) encoding the GFP. In one embodiment, the GFP can be from Aequorea Victoria (U.S. Pat. No. 5,491,084 to Chalfie et al., which are hereby incorporated in their entirety). A plasmid designated pGFP10.1 has been deposited pursuant to, and in satisfaction of, the requirements of the Budapest Treaty on the International Recognition of the Deposit of Microorganisms for the Purposes of Patent Procedure, with the American Type Culture Collection (ATCC), 12301 Parklawn Drive, Rockville, Md. 20852 under ATCC Accession No. 75547 on Sep. 1, 1993. This plasmid is commercially available from the ATCC and comprises a cDNA which encodes a green fluorescent protein of Aequorea Victoria as disclosed in U.S. Pat. No. 5,491,084 to Chalfie et al., which is hereby incorporated in its entirety. A mutated form of this GFP (a red-shifted mutant form) designated pRSGFP-C1 is commercially available from Clontech Laboratories, Inc. (Palo Alto, Calif.).

The plasmid designated pTα1-RSGFP has been deposited pursuant to, and in satisfaction of, the requirements of the Budapest Treaty on the International Recognition of the Deposit of Microorganisms for the Purposes of Patent Procedure, with the American Type Culture Collection (ATCC), 12301 Parklawn Drive, Rockville, Md. 20852 under ATCC Accession No. 98298 on Jan. 21, 1997. This plasmid uses the red shifted GFP (RS-GFP) of Clontech Laboratories, Inc. (Palo Alto, Calif.), and the Tα1 promoter sequence provided by Dr. F. Miller (Montreal Neurological Institute, McGill University, Montreal, Canada). In accordance with the subject invention, the Tα1 promoter can be replaced with another specific promoter, and the RS-GFP gene can be replaced with another form of GFP, by using standard restriction enzymes and ligation procedures.

Mutated forms of GFP that emit more strongly than the native protein, as well as forms of GFP amenable to stable translation in higher vertebrates, are now available and can be used for the same purpose. The plasmid designated pTα1-GFPh has been deposited pursuant to, and in satisfaction of, the requirements of the Budapest Treaty on the International Recognition of the Deposit of Microorganisms for the Purposes of Patent Procedure, with the American Type Culture Collection (ATCC), 12301 Parklawn Drive, Rockville, Md. 20852 under ATCC Accession No. 98299 on Jan. 21, 1997. This plasmid uses the humanized GFP (GFPh) of Zolotukhin and Muzyczka (Levy, J., et al., Nature Biotechnol. 14:610-614 (1996), which is hereby incorporated in its entirety), and the Tα1 promoter sequence provided by Dr. F. Miller (Montreal). In accordance with the subject invention, the Tα1 promoter can be replaced with another specific promoter, and the GFPh gene can be replaced with another form of GFP, by using standard restriction enzymes and ligation procedures. Any nucleic acid molecule encoding a fluorescent form of GFP can be used in accordance with the subject invention.

Other suitable marker proteins include lacZ/beta-galactosidase or alkaline phosphatase.

Standard techniques are then used to place the nucleic acid molecule encoding GFP under the control of the chosen cell specific promoter. Generally, this involves the use of restriction enzymes and ligation.

The resulting construct, which comprises the nucleic acid molecule encoding the GFP under the control of the selected promoter (itself a nucleic acid molecule) (with other suitable regulatory elements if desired), is then introduced into a plurality of cells which are to be sorted. Techniques for introducing the nucleic acid molecules of the construct into the plurality of cells may involve the use of expression vectors which comprise the nucleic acid molecules. These expression vectors (such as plasmids and viruses) can then be used to introduce the nucleic acid molecules into the plurality of cells.

Various methods are known in the art for introducing nucleic acid molecules into host cells. These include: 1) microinjection, in which DNA is injected directly into the nucleus of cells through fine glass needles; 2) dextran incubation, in which DNA is incubated with an inert carbohydrate polymer (dextran) to which a positively charged chemical group (DEAE, for diethylaminoethyl) has been coupled. The DNA sticks to the DEAE-dextran via its negatively charged phosphate groups. These large DNA-containing particles stick in turn to the surfaces of cells, which are thought to take them in by a process known as endocytosis. Some of the DNA evades destruction in the cytoplasm of the cell and escapes to the nucleus, where it can be transcribed into RNA like any other gene in the cell; 3) calcium phosphate coprecipitation, in which cells efficiently take in DNA in the form of a precipitate with calcium phosphate; 4) electroporation, in which cells are placed in a solution containing DNA and subjected to a brief electrical pulse that causes holes to open transiently in their membranes. DNA enters through the holes directly into the cytoplasm, bypassing the endocytotic vesicles through which they pass in the DEAE-dextran and calcium phosphate procedures (passage through these vesicles may sometimes destroy or damage DNA); 5) liposomal mediated transformation, in which DNA is incorporated into artificial lipid vesicles, liposomes, which fuse with the cell membrane, delivering their contents directly into the cytoplasm; 6) biolistic transformation, in which DNA is absorbed to the surface of gold particles and fired into cells under high pressure using a ballistic device; and 7) viral-mediated transformation, in which nucleic acid molecules are introduced into cells using viral vectors. Since viral growth depends on the ability to get the viral genome into cells, viruses have devised efficient methods for doing so. These viruses include retroviruses, lentivirus, adenovirus, herpesvirus, and adeno-associated virus.

As indicated, some of these methods of transforming a cell require the use of an intermediate plasmid vector. U.S. Pat. No. 4,237,224 to Cohen and Boyer, which is hereby incorporated in its entirety, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture. The DNA sequences are cloned into the plasmid vector using standard cloning procedures known in the art, as described by Sambrook et al., Molecular Cloning: A Laboratory Manual 2d Edition, Cold Spring Harbor Laboratory Press, Cold Spring Harbor, N.Y. (1989), which is hereby incorporated in its entirety.

In accordance with one of the above-described methods, the nucleic acid molecule encoding the GFP is thus introduced into a plurality of cells. The promoter which controls expression of the GFP, however, only functions in the cell of interest. Therefore, the GFP is only expressed in the cell of interest. Since GFP is a fluorescent protein, the cells of interest can therefore be identified from among the plurality of cells by the fluorescence of the GFP.

Any suitable means of detecting the fluorescent cells can be used. The cells may be identified using epifluorescence optics, and can be physically picked up and brought together by Laser Tweezers (Cell Robotics Inc., Albuquerque, N. Mex.). They can be separated in bulk through fluorescence activated cell sorting, a method that effectively separates the fluorescent cells from the non-fluorescent cells.

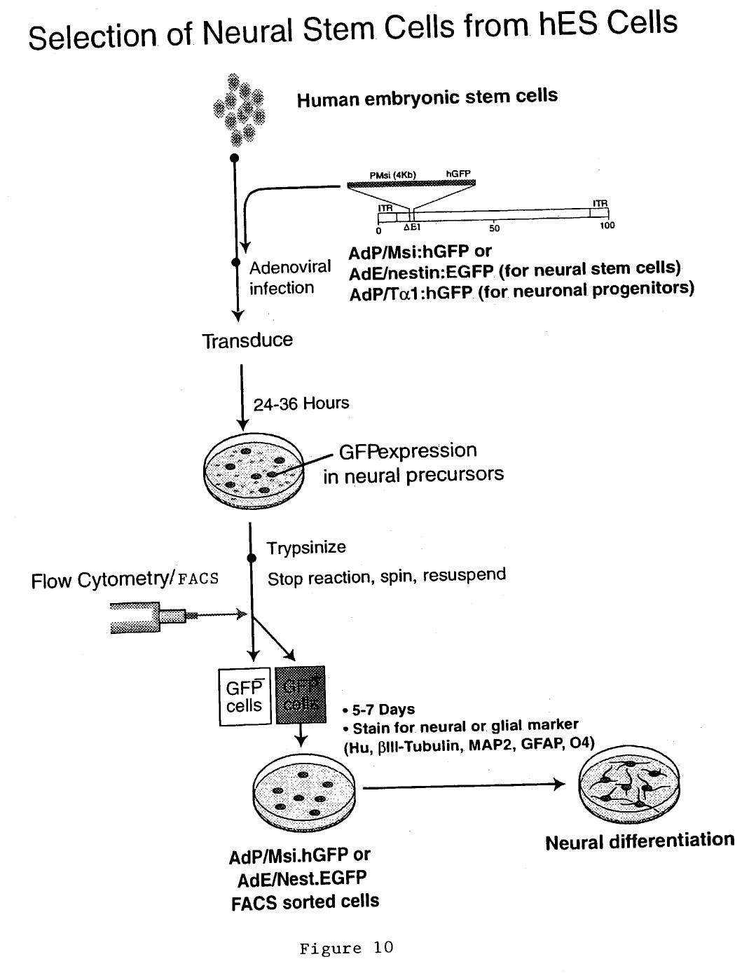

FIG. 10 is a schematic drawing showing an embodiment of the method of the present application. As shown, human embryonic stem cells are subjected to adenoviral infection using the AdP/Msi (i.e. musashi promoter):hGFP or AdE/nestin:EGFP (both for recovery of neural stem cells), or the AdP/Tα1:hGFP (for neuronal progenitor cell recovery) constructs. In a period of 24 to 36 hours after transduction, GFP expression in neural precursons occurs. The cells are then trypsinized, the reaction is stopped, and the cells are spun and resuspended. GFP⁻ and GFP⁺ cells are then recovered using FACS. The GFP⁺ cells are cultured for 5-7 days and stained for neural or glial markers (e.g., Hu, βIII-Tubulin, MAP-2, GFAP, or O4). As a result, AdP/Msi:hGFP, AdE/nestin:EGFP, or AdP/Tα1:hGFP sorted cells are recovered, and these cells undergo neural differentiation.

Desirably, the separated cells are transplanted into a subject. This is carried out by: (1) transuterine fetal intraventricular injection; (2) intraventricular or intraparenchymal (i.e. brain, brain stem, or spinal cord) injections; (3) intraparenchymal injections into adult or juvenile subjects; or (4) intravascular administration. Such administration involves cell doses ranging from 5×10³ to 5×10⁷.

The population of embryonic stem cells can be in a cell culture and is preferably of human origin.

In another embodiment, the present invention relates to a method of isolating neuronal progenitor cells, oligodendrocyte progenitor cells, or neural stem cells from a population of embryonic stem cells by providing a population of embryonic stem cells and differentiating the population of embryonic stem cells to produce a mixed population of cells comprising neuronal progenitor cells, oligodendrocyte progenitor cells, or neural stem cells. A promoter which functions only said neuronal progenitor cells, oligodendrocyte progenitor cells, or neural stem cells is selected and a nucleic acid molecule encoding a marker protein under control of the promoter is introduced into the mixed population of cells. The neuronal progenitor cells, oligodendrocyte progenitor cells, or neural stem cells are then allowed to express the marker protein. Cells expressing the marker protein are separated from the mixed population of cells, where the separated cells are the neuronal progenitor cells, oligodendrocyte progenitor cells, or neural stem cells.

In this embodiment of the present invention, differentiation of the embryonic stem cells is carried out by maintaining the hES cells in a series of culture conditions and factors known to induce selective differentiation to neurons or glial cells namely astrocytes and oligodendrocytes. The first step is to induce the formation of embryoid bodies in non-adherent cultures conditions. For selective induction of neuronal differentiation, the cells are cultured in the presence of neurotrophic factors, like BDNF and NT3, that induce neuronal differentiation as well as survival. Differentiation to astrocytes is achieved by culturing the cells in the presence of serum and to oligodendrocytes in the presence of pro-oligodendrocyte factors like PDGF, bFGF, and triiodothyronine (“T3”).

Another aspect of the present invention is an enriched or purified preparation of isolated oligodendrocyte progenitor cells derived from embryonal stem cells.

A further embodiment of the present invention relates to an enriched or purified preparation of isolated neuronal progenitor cells derived from embryonal stem cells.

Yet an additional aspect of the present invention is directed to an enriched or purified preparation of isolated neural stem cells derived from embryonal stem cells.

### EXAMPLES

### Example 1

Source of hES Cells.

The hES cells were derived from the H9 line (Amit et al., “Clonally Derived Human Embryonic Stem Cell Lines Maintain Pluripotency and Proliferative Potential for Prolonged Periods of Culture,” Dev. Biol. 227:271-278 (2000), which is hereby incorporated by reference in its entirety). They were obtained from Geron Corp. at passage 32.

### Example 2

Culturing of hES Cells.

Cells were maintained and passaged in feeder-free cultures as per published protocols (Carpenter et al., “Enrichment of Neurons and Neural Precursors From Human Embryonic Stem Cells,” Exp. Neurol. 172:383-97 (2001), which is hereby incorporated by reference in its entirety).

Conditioned medium from embryonic mouse fibroblast cells: Fibroblast cells, obtained from E14 mouse embryos, are grown to confluency in gelatin coated flasks. The cells were collected by trypsinization and irradiated at 4000 rads. The irradiated cells were re-plated on gelatin coated flasks at a density of 8 million cells /T175 flask and fed every 24 hrs with Knockout DMEM supplemented with 20% Knockout Serum (Gibco) and bFGF (4ng/ml; Gibco). Conditioned medium was collected every 24 hrs and used to feed the hES cells.

Passaging and maintenance of hES cells: Cultures are passaged every 7-10 days. To passage, hES cells were treated with collagenase type IV (220 units/ml) for 10 mins and scraped of the culture dish. The scraped cells were split 1:3-1:4 on Matrigel (Gibco) coated 6 well plates. The hES cells were fed every 24 hrs with fibroblast conditioned medium supplemented with fresh bFGF (4 ng/ml).

### Example 3

Infection of hES Culture With AdE/nestin:EGFP.

hES cultures that were 40-50% confluent were infected (5 pfu/cell) with a replication defective E1A/1B/E3-deleted type 5 adenovirus bearing E/nestin:EGFP as described in Keyoung et al., “Specific Identification, Selection and Extraction of Neural Stem Cells From the Fetal Human Brain,” Nature Biotech. 19:843-850 (2001), which is hereby incorporated by reference in its entirety. Cells in the hES cultures start expressing EGFP 48-72 hrs after infection.

### Example 4

Flow Cytometry and Sorting.

For FACS separation, cells were dissociated from the Matrigel-coated plates using a 1:1 mixture of collagenase type IV and trypsin/EDTA. The dissociated cells were filtered through a 40 μm filtered and FACS-sorted for EGFP-expressing cells on a FACS Vantage SE (Becton-Dickinson) as described (Roy et al., “In Vitro Neurogenesis by Progenitor Cells Isolated From the Adult Human Hippocampus,” Nature Med. 6:271-277 (2000), which is hereby incorporated by reference in its entirety). Uninfected hES cells were used as a control to set the background fluorescence. A false positive rate of 0.1-0.5 was accepted.

### Example 5

Culturing of Sorted Cells.

Sorted cells were sequentially cultured in: (a) DMEM supplemented with 10% fetal bovine serum and 10 nM all-trans retinoic acid (“RA”) (10 μM) for 5 days in low cluster suspension plates; (b) DMEM/F12 supplemented with N2, B-27 (0.5×), EGF (10 ng/ml), bFGF (10 ng/ml), PDGF (1 ng/ml) and IGF (1 ng/ml) for 5 days on laminin coated plates; and (c) DMEM/F12 supplemented with B-27 (1×), BDNF (10 ng/ml) and NT-3 (10 ng/ml) for 10 days on poly-ornithine/fibronectin coated plates. The cultures were then fixed with 4% paraformaldehyde for subsequent phenotypic analysis.

### Example 6

Immunocytochemistry.

Unsorted and sorted cells were stained at different stages in vitro for nestin protein (rabbit anti-human nestin; 1:400; Dr. Okano), βIII-tubulin (mouse; 1:1000; Covance), MAP-2 (rabbit; 1:500; S. Halpain), or glial fibrillary acidic protein (“GFAP”) (mouse; Sternberger) and O4 (1:2, mouse hybridoma culture supernatant; Dr. Bansal).

### Example 7

Nestin Protein Expressing Cells are Abundant in Native hES Cultures.

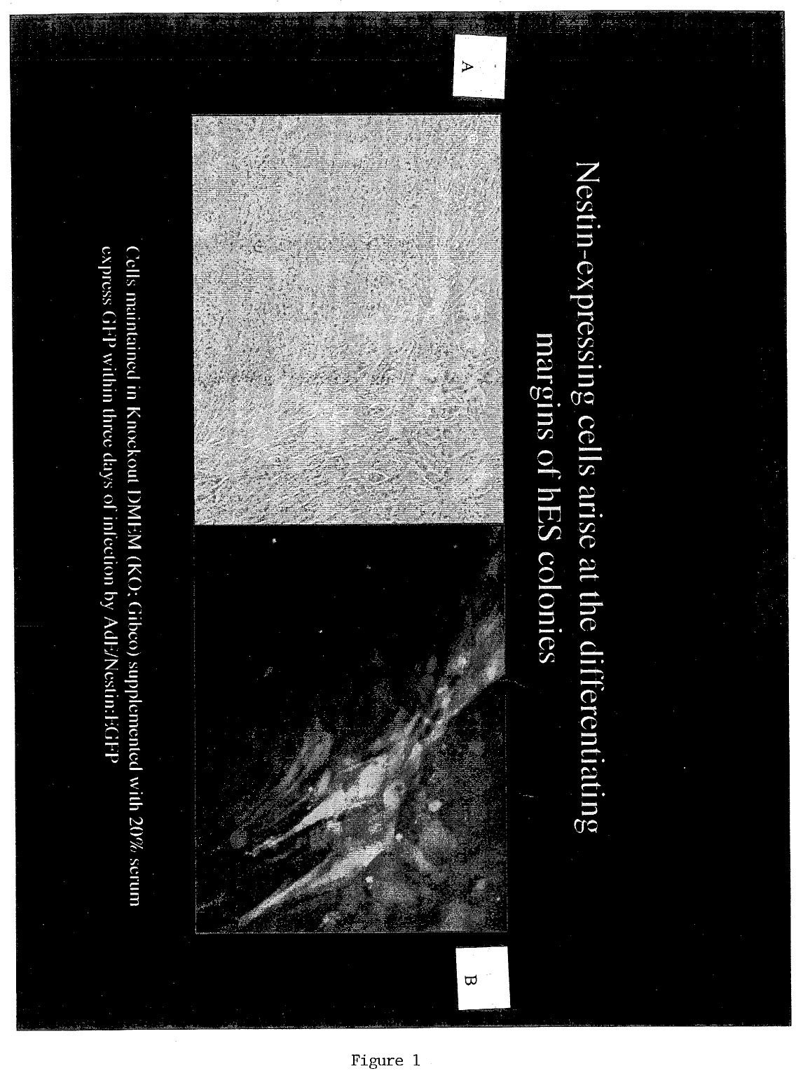

Immunocytochemistry of native hES cultures showed a high number of nestin protein expressing cells. Nestin expression was limited to cells at the edges of individual ES colonies as well as the center of the colonies (FIG. 1). Approximately 30% of the nestin expressing cells incorporated BrdU following a short 2 hr pulse of BrdU in culture prior to fixation, indicating that these cells were actively mitotic. None of the nestin expressing cells co-localized with GFAP, a marker for astrocytes.

### Example 8

E/nestin:EGFP-Positive Cells Constituted a Large Proportion of the hES Population.

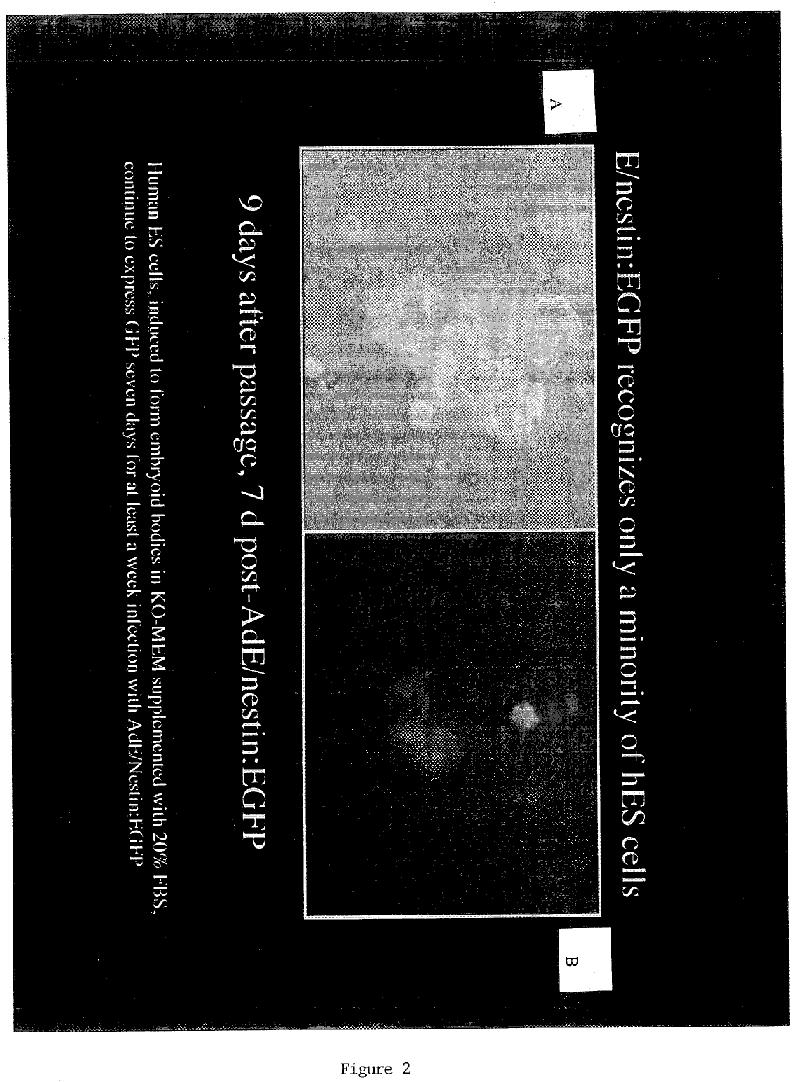

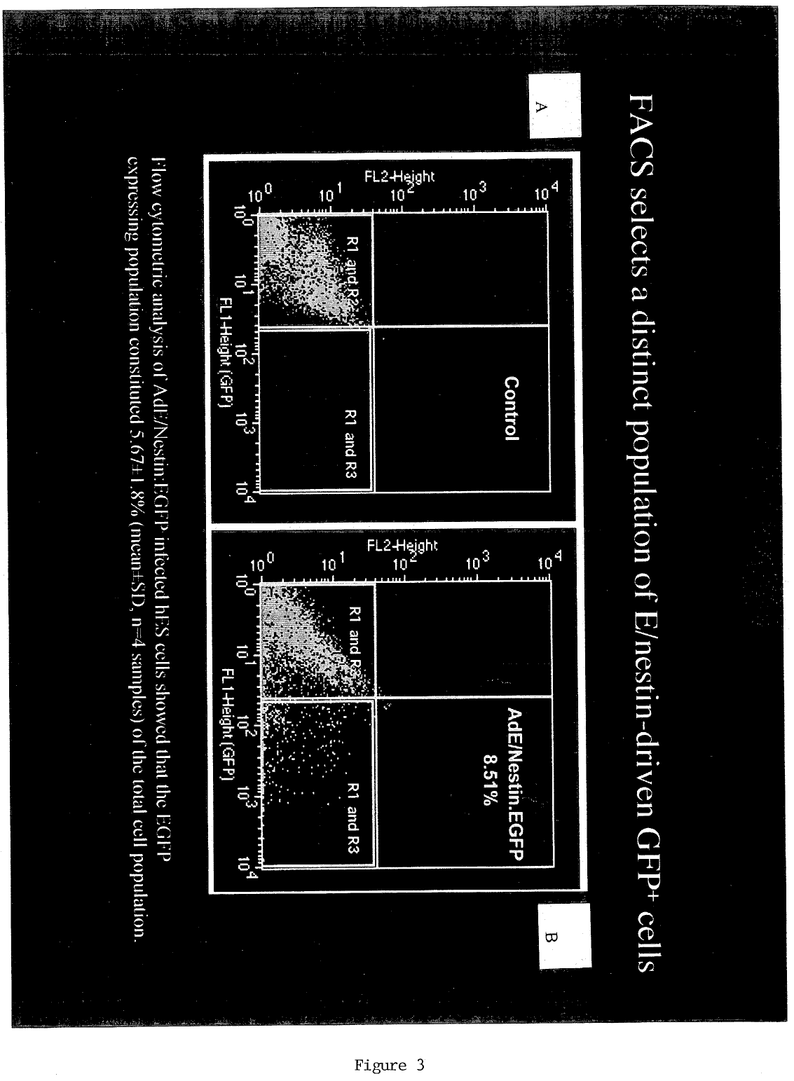

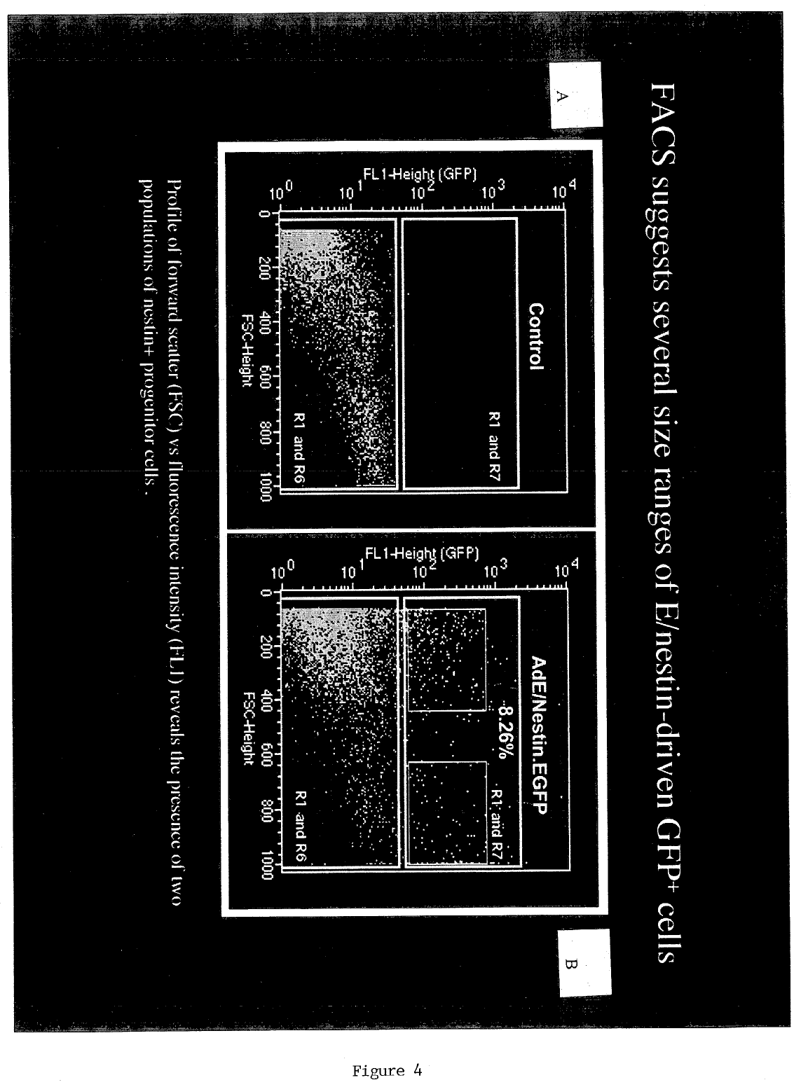

Following infection of hES cells cultured with AdE/nestin:EGFP, EGFP expressing cells were observed 48-72 hrs post-infection. As with the nestin-protein expression profile, EGFP expressing cells were limited to the edge and to the center of the ES colonies. Immunocytochemistry revealed that most of the cells were also expressing nestin protein. Embryoid body formation by AdE/nestin:EGFP-infected hES cells revealed occasional GFP⁺ cells, indicating the residence of neural stem cells within the larger population of cells within each embryoid body (FIG. 2). Flow cytometry analysis of the infected cells showed that the E/nestin-driven EGFP expressing population comprised an average of 5.67±1.8% (mean±SD. n=4 samples) of the total hES population (FIG. 3). These fell into at least 2 readily distinguishable size classes (FIG. 4).

### Example 9

Spheres Generated From E/nestin:EGFP-Sorted Cells Gave Rise to Neurons.

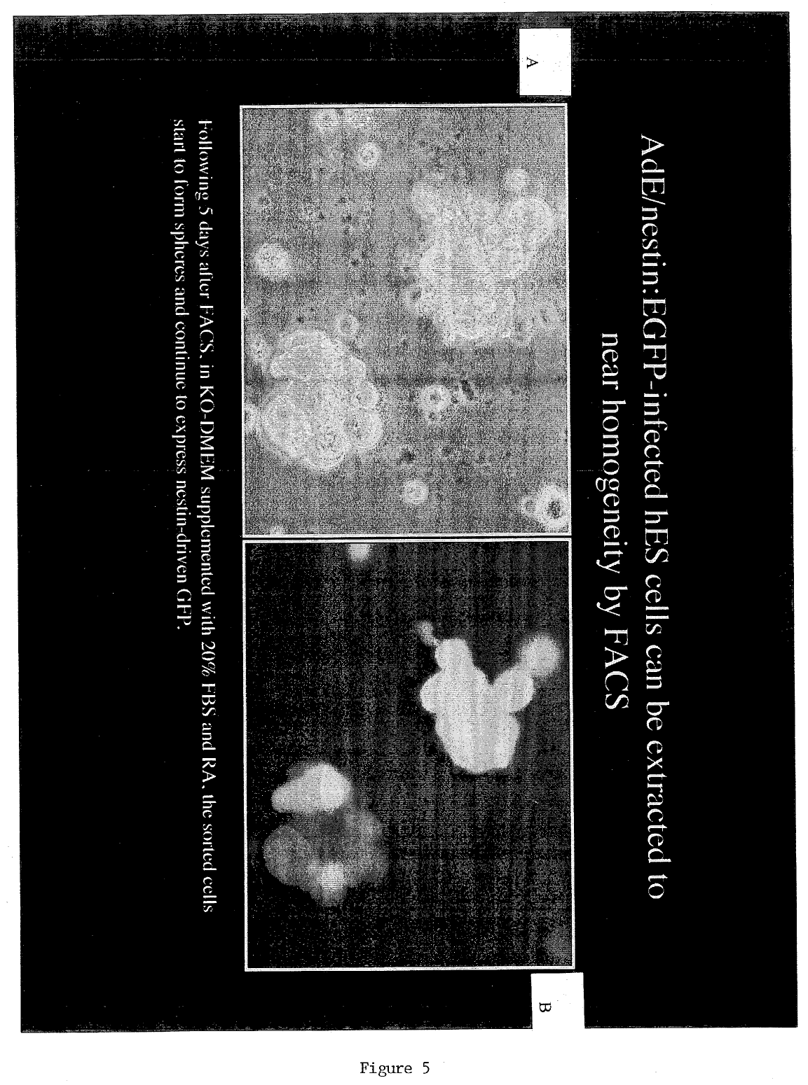

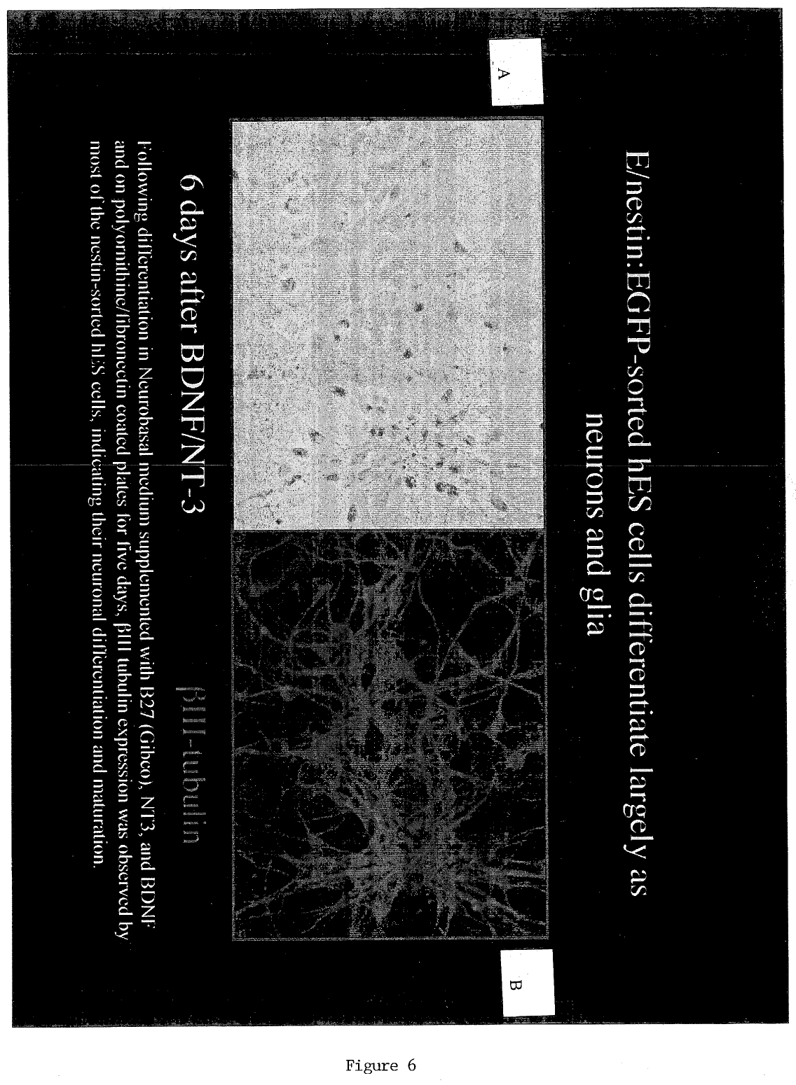

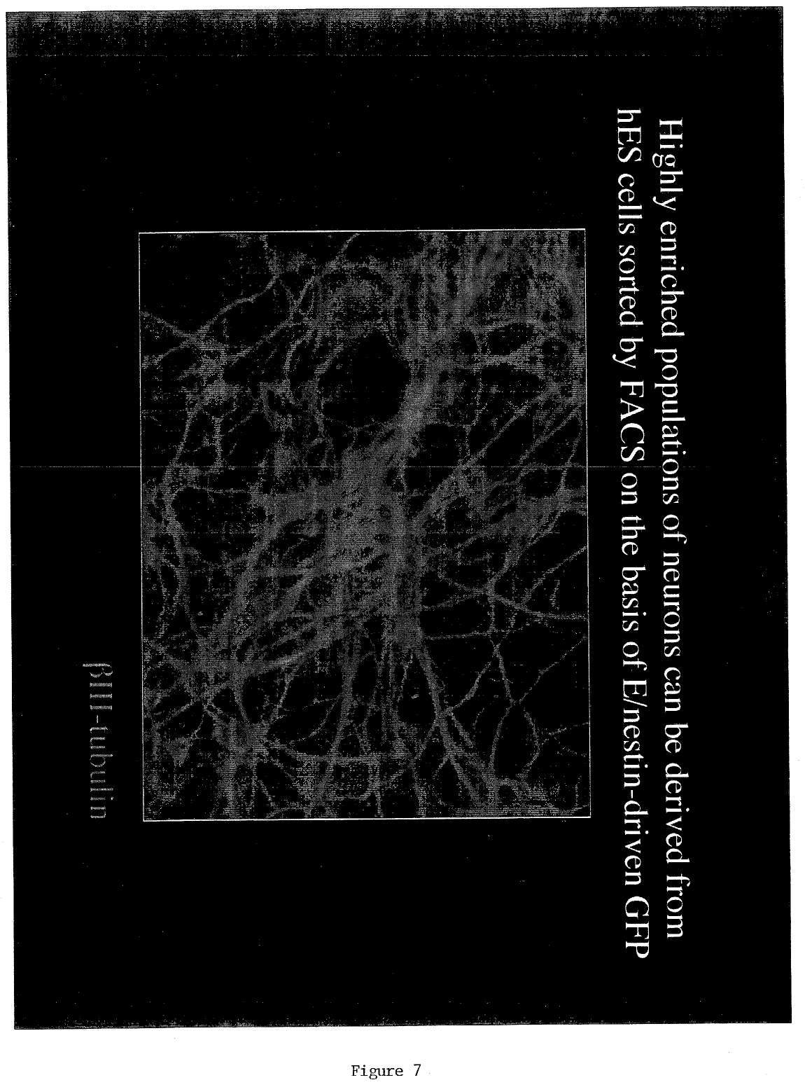

After 4 days in suspension cultures in the presence of RA, small spheres were generated from the E/nestin:EGFP-sorted cells (FIG. 5). These spheres continued to increase in size when cultured in the presence of DMEM/F12 supplemented with B-27, bFGF, EGF, PDGF, and IGF. Following differentiation in BDNF and NT3-supplemented media, on poly-ornithine/fibronectin coated plates for five days, βIII tubulin expression was observed (FIGS. 6 and 7). After 10 days in culture, the βIII tubulin expressing cells matured into MAP-2 expressing neurons.

### Example 10

Individual E/nestin:EGFP-Sorted Cells Were Multipotent.

Limiting dilution, with clonal expansion as neurospheres, revealed that a fraction of AdE/nestin:EGFP-sorted cells were able to both self-renew and co-generate neurons and glia. These hES-derived human neural stem cells could be maintained in continuously expanding culture, with the generation of neurons, glia, and self-replicating neural progenitors. hES-derived neural stem cells (“NSCs”) were able to generate both neurons and glia at all time points during in vitro expansion, and did so to the exclusion of non-neural phenotypes.

### Example 11

Neuronal Progenitor Cells May be Recognized in hES Cultures by Infection With Adenoviruses Encoding P/Tα1 Promoter-Driven hGFP.

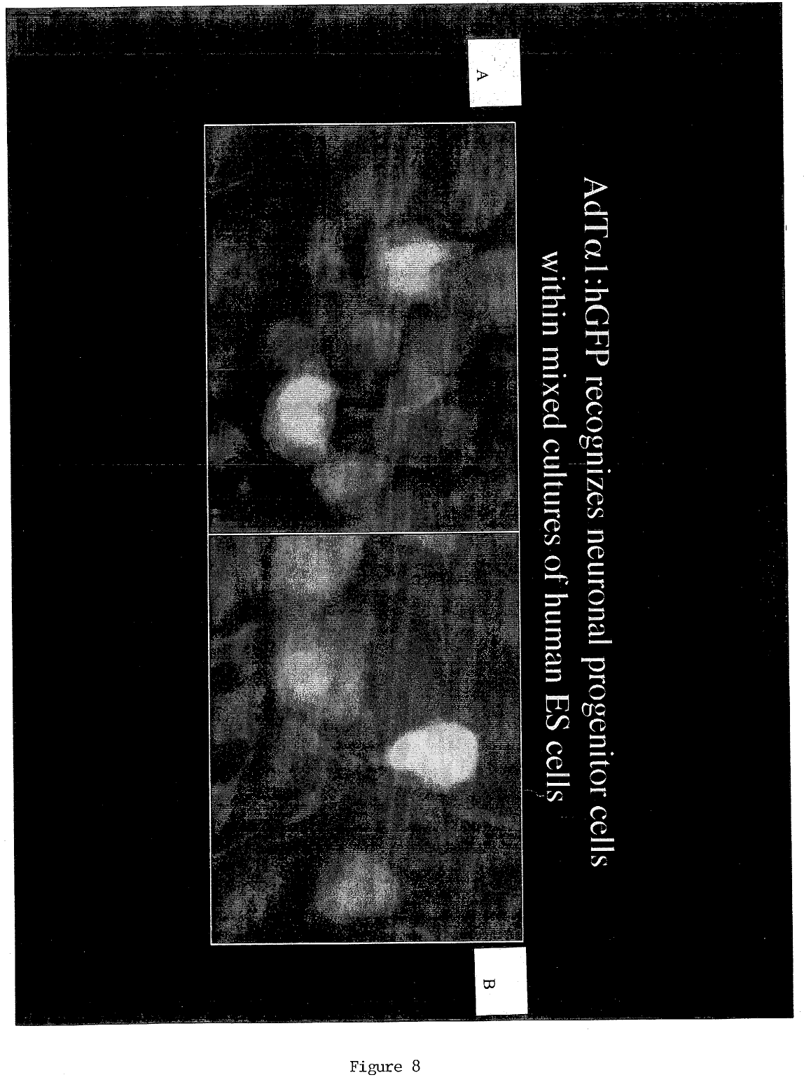

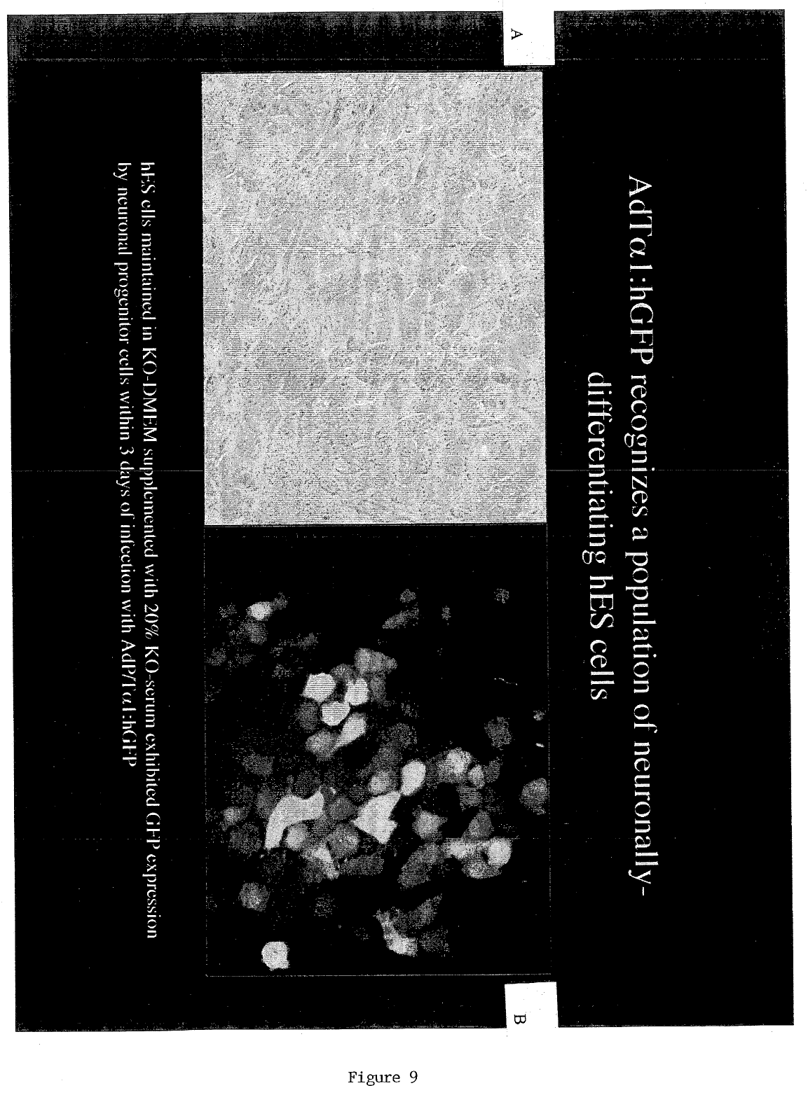

P/Ta1:hGFP may be used to recognize neuronally-committed progenitor cells, themselves the lineage-restricted descendents of E/nestin-defined neural stem cells. On that basis, P/Ta1:hGFP was used to identify and isolate committed neuronal progenitor cells from the mixed ES cell culture (FIGS. 8 and 9). After a week in culture after passage and AdP/Tα1:hGFP viral tagging, neuronally-committed cells were recognized in the larger population of hES cells by their expression of hGFP. This allowed them to then be sorted via FACS.

### Example 12

Lentiviral Construct Containing E/Nestin:EGFP Can be Used to Identify Neural Stem Cells in a Culture of hES Cells.

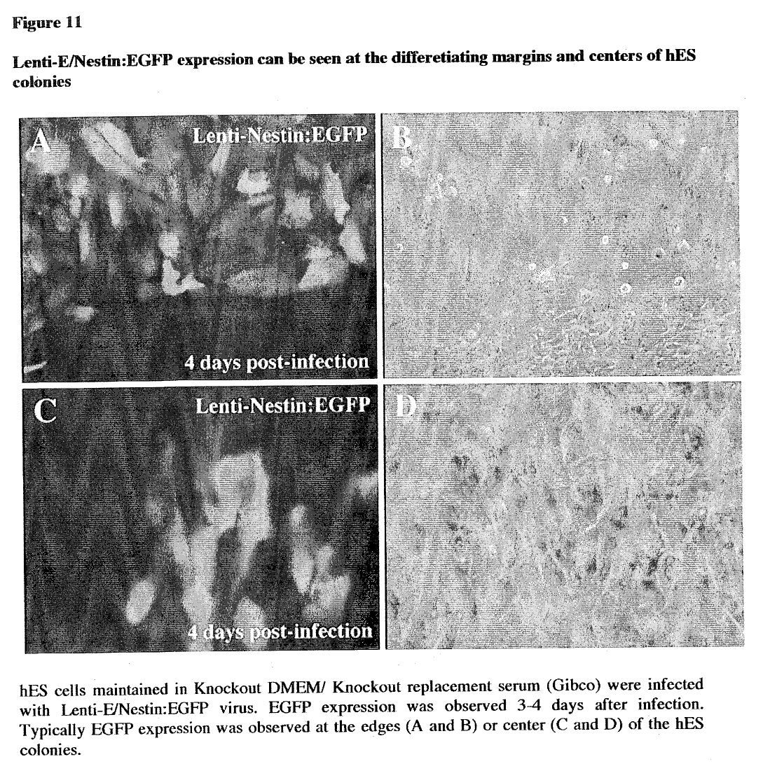

A lentivirus with E/Nestin:EGFP was used to infect cultures of hES cells at a dilution of 1:3000. EGFP expressing cells were observed 48-72 hrs post-infection. As observed with AdE/nestin:EGFP-infected hES cells, the lenti-E/Nestin:EGFP expressing cells were limited to the edge and to the center of the ES colonies (FIG. 11).

### Example 13

EGF Expression by Lenti-E/Nestin:EGFP Infected hES Cells Continues Through Several Generations.

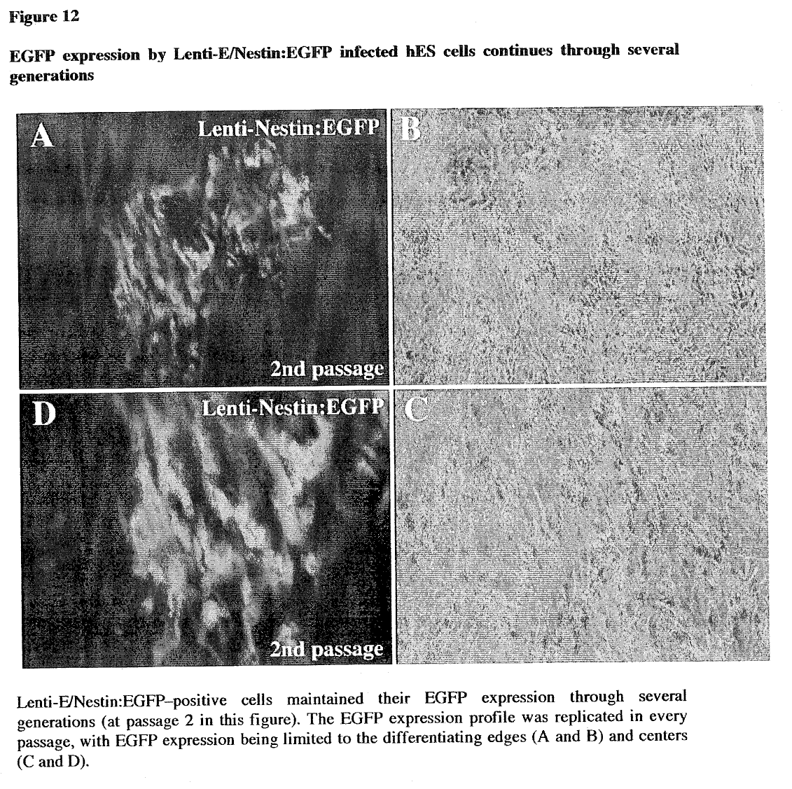

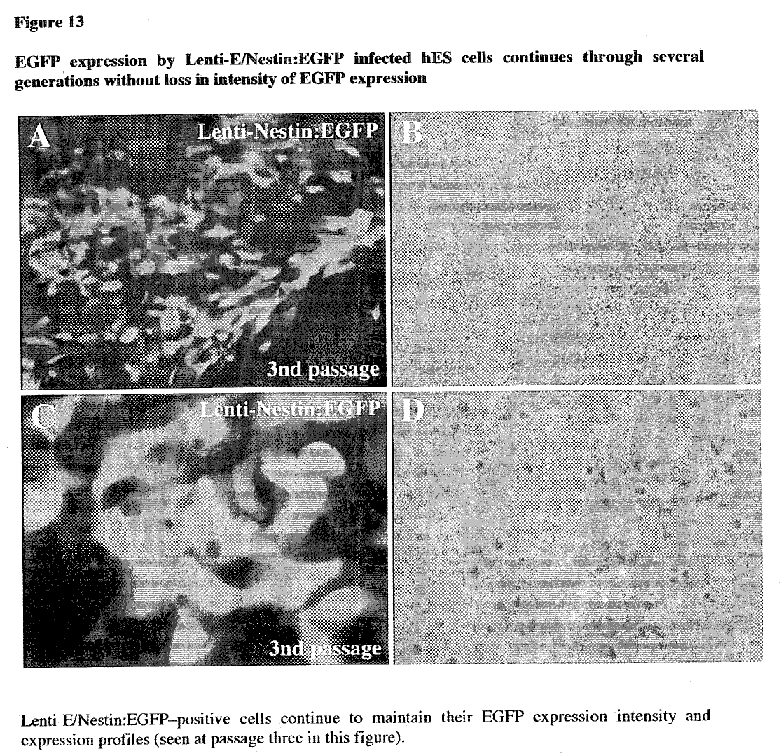

Due the integration of the lenti-E/Nestin:EGFP into the host genome, progeny of the initially infected-EGFP (passage=0) expressing cells continued to express EGF for several passages (FIGS. 12 and 13). No loss in the intensity of EGFP expression was observed.

### Example 14

Lenti-E/Nestin:EGFP Expressing Cells Constitute a Large Proportion of the hES Population.

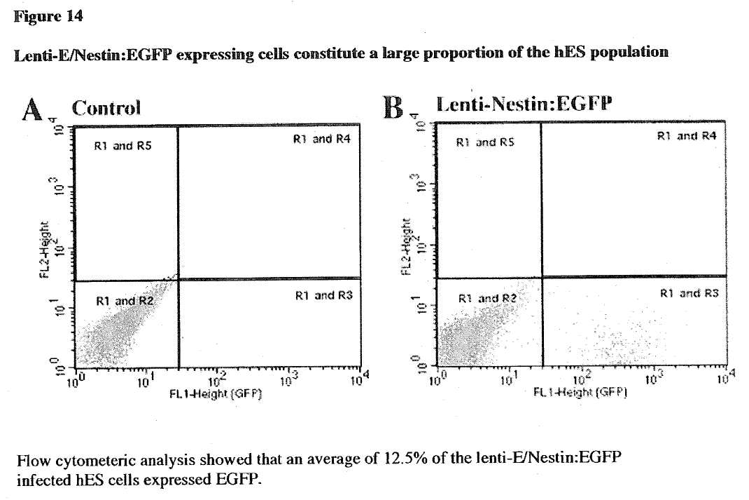

Flow cytometery analysis of the lenti-E/Nestin:EGFP infected cells showed that the EGFP expressing population constituted around 12.5% of the total hES population (FIG. 14).

### Example 15

Spheres Generated from Lenti-E/Nestin:EGFP-sorted Cells Gave Rise to Neurons and Glia.

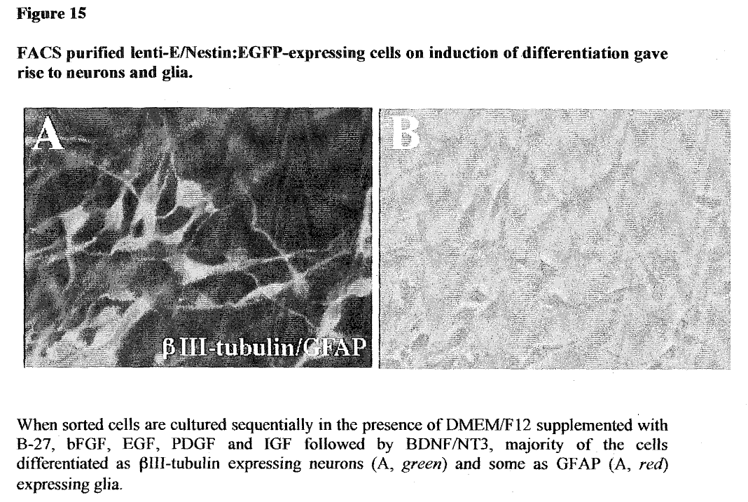

Lenti-E/Nestin:EGFP-sorted cells generated spheres after being cultured in suspension culture dishes for 4 days in the presence of RA. These spheres were then cultured in the presence of DMEM/F12 supplemented with B-27, bFGF, EGF, PDGF, and IGF. The sorted cells were then maintained in BDNF/NT3 supplemented neurobasal medium on poly-ornithine/fibronecting coated culture dishes. After 3 weeks, the majority of the cells differentiated as βIII-tubulin expressing neurons and some as GFAP expressing glia (FIG. 15).

### Example 16

Lenti-Tα1:hGFP Recognizes Neuronal Progenitors in Mixed hES Cultures.

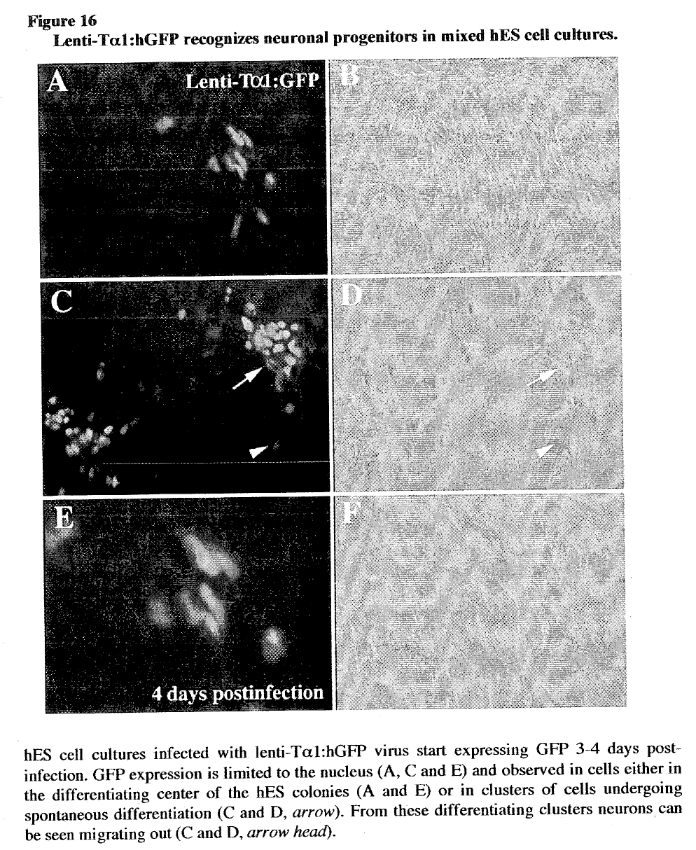

A lentivirus with E/Nestin:EGFP was used to infect cultures of hES cells at a dilution of 1:300. GFP expressing cells were observed 3-4 days post-infection. The GFP expression was primarily limited to the nucleus and observed in cells either in the center of the hES colonies or in clusters of cells undergoing spontaneous differentiation (FIG. 16).

### Example 17

Lenti-Tα1:hGFP is Expressed by a Significant Proportion of the hES Population.

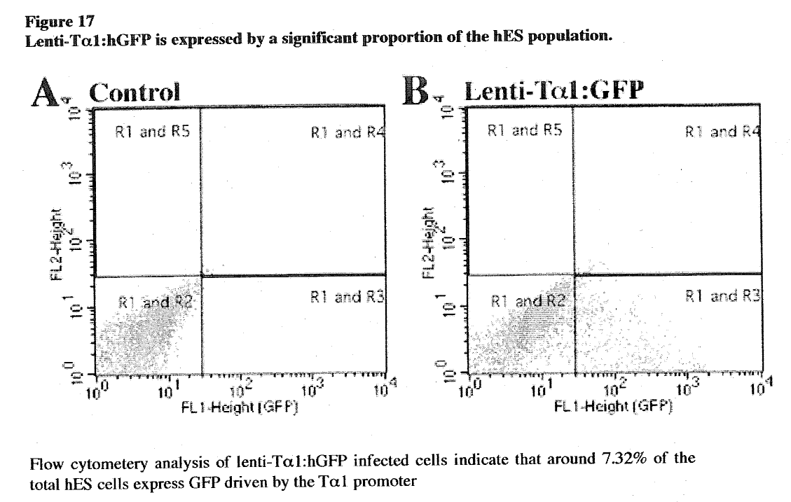

Flow cytometery analysis of the lenti-Ta1:hGFP infected cells showed that around 7.32% of the total hES expressed GFP driven by the Tα1 promoter (FIG. 17).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of isolating neuronal progenitor cells, oligodendrocyte progenitor cells, or neural stem cells from a population of embryonic stem cells comprising:
selecting a promoter which functions only in said neuronal progenitor cells, oligodendrocyte progenitor cells, or neural stem cells;
introducing a nucleic acid molecule encoding a marker protein under control of said promoter into the population of embryonic stem cells;
differentiating the population of embryonic stem cells to produce a mixed population of cells comprising neuronal progenitor cells, oligodendrocyte progenitor cells, or neural stem cells;
allowing the neuronal progenitor cells, oligodendrocyte progenitor cells, or neural stem cells to express the marker protein; and
separating the cells expressing the marker protein from the mixed population of cells, wherein said separated cells are said neuronal progenitor cells, oligodendrocyte progenitor cells, or neural stem cells.

2. The method of claim 1, wherein said introducing comprises viral mediated transduction of the population of embryonic stem cells.

3. The method of claim 2, wherein said viral mediated transduction comprises adenovirus-mediated transduction, retrovirus-mediated transduction, lentivirus-mediated transduction, or adeno-associated virus-mediated transduction.

4. The method of claim 1, wherein said introducing comprises electroporation.

5. The method of claim 1, wherein said introducing comprises biolistic transformation.

6. The method of claim 1, wherein said introducing comprises liposomal mediated transformation.

7. The method of claim 1, wherein the marker protein is a fluorescent protein and said separating comprises fluorescence activated cell sorting.

8. The method of claim 1, wherein the marker protein is either lacZ/beta-galactosidase or alkaline phosphatase.

9. The method of claim 1, wherein neuronal progenitor cells are separated from the mixed population and said promoter is a Tα1 tubulin promoter, a Hu promoter, an ELAV promoter, a MAP-1B promoter, or a GAP-43 promoter.

10. The method of claim 1, wherein oligodendrocyte progenitor cells are separated from the mixed population and said promoter is a CNP promoter, an NCAM promoter, a myelin basic protein promoter, a JC virus minimal core promoter, a myelin-associated glycoprotein promoter, or a proteolipid protein promoter.

11. The method of claim 1, wherein neural stem cells are separated from the mixed population and said promoter is the musashi promoter or the nestin enhancer.

12. The method according to claim 1 further comprising:
identifying the cells of said mixed population of cells that express the marker protein, wherein the identifying step is after the allowing step.

13. The method of claim 1, wherein the population of embryonic stem cells is in a cell culture.

14. The method of claim 1 further comprising:
transplanting the separated cells into a subject.

15. The method of claim 1, wherein the neuronal progenitor cells, oligodendrocyte progenitor cells, and neural stem cells are of human origin.

16. An enriched population of neuronal progenitor cells isolated by the method of claim 1.

17. An enriched population of oligodendrocyte progenitor cells isolated by the method of claim 1.

18. An enriched population of neural stem cells produced by the method of claim 1.

19. A method of isolating neuronal progenitor cells, oligodendrocyte progenitor cells, or neural stem cells from a population of embryonic stem cells comprising:
providing a population of embryonic stem cells;
differentiating the population of embryonic stem cells to produce a mixed population of cells comprising neuronal progenitor cells, oligodendrocyte progenitor cells, or neural stem cells;
selecting a promoter which functions only in said neuronal progenitor cells, oligodendrocyte progenitor cells, or neural stem cells;
introducing a nucleic acid molecule encoding a marker protein under control of said promoter into the mixed population of cells;
allowing the neuronal progenitor cells, oligodendrocyte progenitor cells, or neural stem cells to express the marker protein; and
separating the cells expressing the marker protein from the mixed population of cells, wherein said separated cells are said neuronal progenitor cells, oligodendrocyte progenitor cells, or neural stem cells.

20. The method of claim 19, wherein said introducing comprises viral mediated transduction of the mixed population of cells.

21. The method of claim 20, wherein said viral mediated transduction comprises adenovirus-mediated transduction, retrovirus-mediated transduction, lentivirus-mediated transduction, or adeno-associated virus-mediated transduction.

22. The method of claim 19, wherein said introducing comprises electroporation.

23. The method of claim 19, wherein said introducing comprises biolistic transformation.

24. The method of claim 19, wherein said introducing comprises liposomal mediated transformation.

25. The method of claim 19, wherein the marker protein is a fluorescent protein and said separating comprises fluorescence activated cell sorting.

26. The method of claim 19, wherein the marker protein is either lacZ/beta-galactosidase or alkaline phosphatase.

27. The method of claim 19, wherein neuronal progenitor cells are separated from the mixed population and said promoter is a Tα1 tubulin promoter, a Hu promoter, an ELAV promoter, a MAP-1B promoter, or a GAP-43 promoter.

28. The method of claim 19, wherein oligodendrocyte progenitor cells are separated from the mixed population and said promoter is a CNP-P2 promoter, a CNP-P1+P2 promoter, an NCAM promoter, a myelin basic protein promoter, a JC virus minimal core promoter, a myelin-associated glycoprotein promoter, or a proteolipid protein promoter.

29. The method of claim 19, wherein neural stem cells are separated from the mixed population and said promoter is the musashi promoter or the nestin enhancer.

30. The method according to claim 19, further comprising:
identifying the cells of said mixed population of cells that express the marker protein, wherein the identifying step is after the allowing step.

31. The method of claim 19 further comprising:
transplanting the separated cells into a subject.

32. The method of claim 19, wherein the neuronal progenitor cells, oligodendrocyte progenitor cells, and neural stem cells are of human origin.

33. An enriched population of neuronal progenitor cells produced by the process of claim 19.

34. An enriched population of oligodendrocyte progenitor cells produced by the process of claim 19.

35. An enriched population of neural stem cells produced by the process of claim 19.

36. An enriched or purified preparation of isolated oligodendrocyte progenitor cells derived from embryonal stem cells.

37. The enriched or purified preparation of isolated oligodendrocyte progenitor cells of claim 36 which are human.

38. An enriched or purified preparation of isolated neuronal progenitor cells derived from embryonal stem cells.

39. The enriched or purified preparation of isolated neuronal progenitor cells of claim 38 which are human.

40. An enriched or purified preparation of isolated neural stem cells derived from embryonal stem cells.

41. The enriched or purified preparation of isolated neural stem cells of claim 40 which are human.
